(19) United States
(12) Patent Application Publication
(10) Pub. No.: US 2012/0097171 A1
(43) Pub. Date: Apr. 26, 2012

(54) INHIBITOR OF PATHOGENS

(76) Inventor: Maurice Roger POPE, Kwa Zulu Natal (ZA)

(21) Appl. No.: 13/281,579

(22) Filed: Oct. 26, 2011

(30) Foreign Application Priority Data
Oct. 26, 2010 (NZ) 588785

(51) Int. Cl. A61F 6/06

(52) U.S. Cl. 128/830

(57) ABSTRACT

An intravaginal device for use in combating pathogens includes a ring of flexible material shaped to be received and retained in the vaginal cavity and includes at least one strand of copper wire.

### BACKGROUND TO THE INVENTION

Many Sexually transmitted pathogens are starting to show resistance to antibiotics that were previously effective in their control.

Copper has been shown to be effective against a number of pathogens, including those responsible for Sexually Transmitted Infections. (STI's). In the U.S., the Environmental Protection Agency regulates the registration of antimicrobial products. After extensive antimicrobial testing according to the Agency's stringent test protocols, 355 copper alloys, including many brasses, were found to kill more than 99.9% of methicillin-resistant Staphylococcus aureus (MRSA), E. coli O157:H7, Pseudomonas aeruginosa, Staphylococcus aureus, Enterobacter aerogenes, and vancomycin-resistant Enterococci within two hours of contact.

A number of Copper Intrauterine Devices (IUDs) such as the Copper T contain copper. It has been found that the copper negatively affects the mobility of sperm and prevents them from joining with an egg, and has the added advantage of proving fatal to Gonococcal bacteria.

However, an IUD must be inserted by a Medical Doctor, and while modern day IUD's are much safer than in the past, they still pose some risks. Most doctors prefer to use IUDs only in women who have already had a baby. Side effects of all IUDs include cramping or pain when the IUD is first inserted and spotting between periods for the first 3 to 6 months. Side effects of copper IUDs include heavier bleeding and stronger cramps during periods.

In recent years, a contraceptive device known as “the ring” (NuvaRing) has been developed. This is a small, flexible intravaginal device that is replaced on a monthly basis. The ring releases combined hormones (estrogen and progestin) to protect against pregnancy. The user is able to insert and remove the device herself. While proving to be a competent method of contraception, this device does not provide any protection against STI's.

It is an object of this invention to provide a device which, at least partially, alleviates some of the abovementioned problems.

### SUMMARY OF THE INVENTION

In accordance with this invention, there is provided an intravaginal device for use in combating pathogens, the device comprising a flexible material shaped to be received and retained in the vaginal cavity and including one or more copper containing elements.

The copper containing element is preferably a strand of copper wire.

The strands of copper wire may be wound around the flexible material, embedded in the flexible material, or located alongside the flexible material.

The flexible material may include rubber, preferably silicon rubber.

The device may further include an additional metal element capable of acting as an anode to the copper. The metal element may include zinc.

Alternatively, the device may include a copper alloy, such as brass.

The device may be circular or ring shaped.

In one embodiment of the invention, the device may comprise a ring of silicon rubber tubing, around which one or more strands of copper or a copper alloy may be wound.

In another embodiment of the invention, the device may comprise alternating sections of silicon rubber tubing and copper or copper alloy windings linked to one another to form a ring.

In a preferred embodiment of the invention the copper windings are positioned on opposite sides of the ring formation.

Additional medicinal substances such as antibiotic formulations or hormonal substances may be added to the device.

### BRIEF DESCRIPTION OF THE DRAWINGS

A preferred embodiment of the invention is described below by way of example only and with reference to the following drawings, in which;

FIG. 1 is a representation of the intravaginal device.

### DETAILED DESCRIPTION OF THE INVENTION

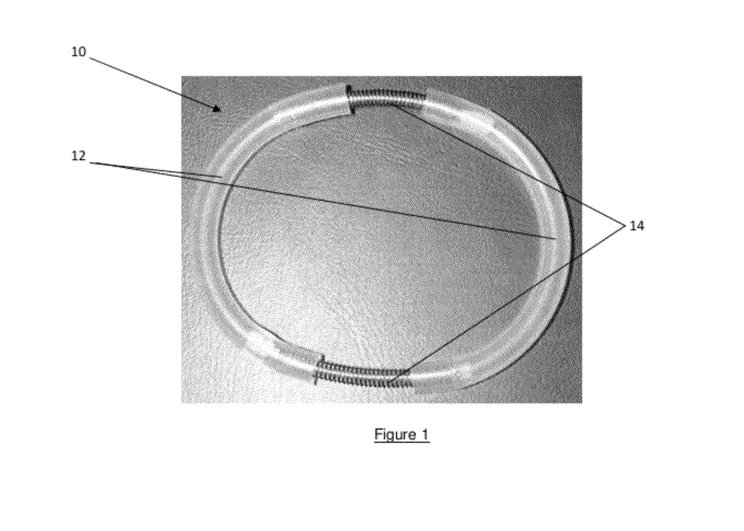

Referring to FIG. 1 the intravaginal device 10 for use in combating pathogens includes two lengths of flexible silicon rubber tubing 12, joined together by two sections of copper windings 14 to form a ring.

The copper windings 14 are positioned at opposite sides of the ring so that the copper ions released by dissolution by the fluids secreted within the vagina, create an electromagnetic field.

In a second embodiment of the invention, the potency of the copper against undesired pathogens may be increased by substituting one of the copper windings for a copper alloy such as brass. Brass is a combination of Copper and Zinc, and in this instance, the zinc acts as a sacrificial anode, to increase the ionisation of the copper. Alternatively, an additional metal element capable of acting as an anode to copper may be separately introduced to the metal ring. The metal element could include zinc, the ions of which enhance those of copper in the spectrum of pathogen inhibitors.

In use, the device is inserted into the vagina after a menstrual period and left in place until the next menstrual period, at which time it may be removed and sterilised. It is believed that the copper present in the device will aid in the prevention of infection by a wide range of pathogens. In particular, the copper present may prove fatal to those pathogens responsible for Sexually Transmitted Infections that display drug resistant tendencies, such as Gonococcal bacteria.

In addition, it is anticipated that the electromagnetic field created by the copper ions may attract sperm to the device, thus affecting the mobility of the sperm and preventing them from moving into the uterus and fallopian tubes. In this way the device will act as a pathogen inhibitor, and may possibly also act as a contraceptive device.

Numerous modifications to this embodiment are possible, without departing from the scope of the invention. For example, the strands of copper wire may be embedded in the flexible material, additional medicinal substances such as antibiotic formulations or hormonal substances may be added to the device. The additional substances may either be added as a separate clip-on, attachable to the device. Alternatively, the additional substances may be impregnated in the device.

The invention therefor provides a novel inhibitor of sexually transmitted pathogens.

1. An intravaginal device for use in combating pathogens, the device comprising a flexible material shaped to be received and retained in the vaginal cavity and including one or more copper containing elements.

2. An intravaginal device as claimed in claim 1 in which the flexible material is a rubber compound.

3. An intravaginal device as claimed in claim 2 in which the rubber compound is silicon rubber.

4. An intravaginal device as claimed in claim 1 in which the device includes one or more strands of copper wire.

5. An intravaginal device as claimed in claim 4 in which the strands of copper wire are either wound around the flexible material, embedded in the flexible material, or located alongside the flexible material.

6. An intravaginal device as claimed in claim 1 in which the device includes an additional metal element capable of acting as an anode to the copper.

7. An intravaginal device as claimed in claim 6 in which the metal element includes zinc.

8. An intravaginal device as claimed in claim 6 in which the device includes a copper alloy, such as brass.

9. An intravaginal device as claimed in claim 1 in which the device is circular.

10. An intravaginal device as claimed in claim 1 in which the device is ring shaped.

11. An intravaginal device as claimed in claim 1 in which the device comprises a ring of silicon rubber tubing, around which one or more strands of copper or a copper alloy are wound.

12. An intravaginal device as claimed in claim 1 in which the device comprises alternating sections of silicon rubber tubing and copper or copper alloy windings linked to one another to form a ring.

13. An intravaginal device as claimed in claim 11 in which the copper windings are positioned on opposite sides of the ring formation.

14. An intravaginal device as claimed in claim 1 in which the device is capable of receiving additional medicinal substances such as antibiotic formulations or hormonal substances.

15. An intravaginal device as claimed in claim 2 in which the device includes one or more strands of copper wire.

16. An intravaginal device as claimed in claim 3 in which the device includes one or more strands of copper wire.

17. An intravaginal device as claimed in claim 2 in which the device includes an additional metal element capable of acting as an anode to the copper.

18. An intravaginal device as claimed in claim 3 in which the device includes an additional metal element capable of acting as an anode to the copper.
